(19) United States
(12) Patent Application Publication
(10) Pub. No.: US 2006/0216311 A1
(43) Pub. Date: Sep. 28, 2006

(54) Method and compositions for conferring viral immunity and reversing viral pathogenesis via strategic infection with a theravirus thereby providing genomic integration of genetically engineered, replication incompetent, integrating viral DNA

(76) Inventor: Lawrence F. Glaser, Fairfax Station, VA (US)

Correspondence Address: Donald R. Studebaker;NIXON PEABODY LLP, Suite 900, 401 9th Street, N.W., Washington, DC, 20004, US

(21) Appl. No.: 11/441,860

(22) Filed: May 26, 2006

Related U.S. Application Data
(63) Continuation of application No. 10/298,997, filed on Nov. 19, 2002.
(60) Provisional application No. 60/331,563, filed on Nov. 19, 2001.

(51) Int. Cl. A61K 39/21; C12Q 1/70; C12N 7/00; C12N 15/867

(52) U.S. Cl. 424/208.1; 435/5; 435/456; 435/235.1

(57) ABSTRACT

The present invention provides a method of conferring viral immunity and/or reversing viral pathogenesis via strategic genomic integration of a genetically engineered replication incompetent vDNA composition, i.e., a theravirus. The invention is generally effectuated by administering to a host a therapeutic amount of genetically engineered viral DNA (vDNA) composition in an amount sufficient to out-compete the natural pathogenic vDNA's ability to integrate into the host cells genome. The genetically engineered vDNA construct is genetically altered in a manner that effectively prevents post integration transcription of the composition. The invention is effective in the prevention and treatment of viral infections, including, HIV (AIDS), herpes and hepatitis.

### CROSS REFERENCE TO RELATED APPLICATIONS

This application is a related to U.S. Provisional Application, under 35 U.S.C 119(e) Ser. No. 60/331,563, the entire disclosure of which is hereby incorporated by reference.

### BACKGROUND OF THE INVENTION

HIV and other viral infections such as hepatitis, are globally recognized as but a few of the leading causes of death based upon viral born disease and pathogenicity. HIV is the virus known to cause acquired immunodeficiency syndrome (AIDS) in humans. HIV is a disease in which a virus is replicated in the body or more specifically, in host cells. The virus attacks the body's immune system. As to HIV and AIDs, several drugs have been approved for treatment of this devastating disease pathogenesis, including azidovudine (AZT), didanosine (dideoxyinosine, ddI), d4T, zalcitabine (dideoxycytosine, ddC), nevirapine, lamivudine (epivir, 3TC), saquinavir (Invirase), ritonavir (Norvir), indinavir (Crixivan), and delavirdine (Rescriptor). See M. I. Johnston & D. F. Hoth, Science, 260(5112), 1286-1293 (1993) and D. D. Richman, Science, 272(5270), 1886-1888 (1996). An AIDS vaccine (Salk's vaccine) has been tested and several proteins which are chemokines from CD8 have been discovered to act as HIV suppressers. In addition to the above synthetic nucleoside analogs, proteins, and antibodies, several plants and substances derived from plants have been found to have in vitro anti-HIV activity. However, HIV virus is not easily destroyed nor is there a good mechanism for keeping the host cells from replicating the virus. HIV has diversified its genome to exhibit a mutation capacity and recombination capacity which automatically adjusts, dynamically and contemporaneously, to the drugs or biologicals applied to the infected host. To date, any and all combinations of HIV+ and AIDs therapy merely buy time, however, they also universally promote acceleration of mutation and recombinatorial HIV, causing a more rapid mutagenesis and thus a more rapid diversification of the overall HIV genome. Development of new drugs and biologicals cannot keep pace with the rate at which HIV can dodge any and all combination therapies. Although admittedly, we buy time patient by patient, we also diversify and intensify the core issue, which is to stop HIV permanently in all of its activities (mutation, diversification and infection).

Thus, medical professionals continue to search for drugs that can guard against transmission of HIV infection from host to host, prevent HIV and retroviral infections, treat HIV carriers to prevent their disease from progressing to full-blown deadly AIDS, and to treat the AIDS patient.

Herpes simplex virus (HSV) types 1 and 2 are persistent viruses that commonly infect humans; they cause a variety of troubling human diseases. HSV type 1 causes oral “fever blisters” (recurrent herpes labialis), and HSV type 2 causes genital herpes, which has become a major venereal disease in many parts of the world. No fully satisfactory treatment for genital herpes currently exists. In addition, although it is uncommon, HSV can also cause encephalitis, a life-threatening infection of the brain. (The Merck Manual, Holvey, Ed., 1972; Whitley, Herpes Simplex Viruses, In: Virology, 2nd Ed., Raven Press (1990)).

A most serious HSV-caused disorder is dendritic keratitis, an eye infection that produces a branched lesion of the cornea, which can in turn lead to permanent scarring and loss of vision. Ocular infections with HSV are a major cause of blindness. HSV is also a virus which is difficult, if not impossible to cure. It has been recently noted by Harvard researchers, HSV types, such as type 6, have been pervasively found in viral breakout, as a patient passes from HIV+ to full-blown AIDs.

In either case, researchers are in agreement that viral persistence and presence, attracts a variety of immune system mediated activity, some of which is utterly deleterious to the host cell and thus, to the immune system as a whole. Chronic HIV presents a complicated pathogenesis, as disease progression moves from HIV+ to AIDs, toward wasting of the immune system, immune system incompetence via reduced maturing repertoire, a host of known secondary and tertiary deleterious and morbid disease conditions and ultimately, mortality for the host.

Hepatitis is a disease of the human liver. It is manifested with inflammation of the liver and is usually caused by viral infections and sometimes from toxic agents. Hepatitis may progress to liver cirrhosis, liver cancer, and eventually death. Several viruses such as hepatitis A, B, C, D, E and G are known to cause various types of viral hepatitis. Among them, HBV and HCV are the most serious. HBV is a DNA virus with a virion size of 42 nm. HCV is a RNA virus with a virion size of 30-60 nm. See D. S. Chen, J. Formos. Med. Assoc., 95(1), 6-12 (1996). In either case, researchers are in agreement that viral persistence and presence, attracts a variety of immune system mediated activity, some of which is utterly deleterious to the host cell and thus, to the organ. Chronic Hepatitis presents a complicated pathogenesis, as disease progression moves toward wasting of a crucial organ and thus, mortality for the host.

Hepatitis C infects 4 to 5 times the number of people infected with HIV. Hepatitis C is difficult to treat and it is estimated that there are 500 million people infected with it worldwide (about 15 times those infected with HIV). No effective immunization is currently available, and hepatitis C can only be controlled by other preventive measures such as improvement in hygiene and sanitary conditions and interrupting the route of transmission. At present, the only acceptable treatment for chronic hepatitis C is interferon which requires at least six (6) months of treatment and or ribavarin which can inhibit viral replication in infected cells and also improve liver function in some people. Treatment with interferon however has limited long term efficacy with a response rate of about 25%.

Hepatitis B virus infection can lead to a wide spectrum of liver injury. Moreover, chronic hepatitis B infection has been linked to the subsequent development of hepatocellular carcinoma, a major cause of death. Current prevention of HBV infection is a hepatitis B vaccination which is safe and effective. However, vaccination is not effective in treating those already infected (i.e., carriers and patients). Many drugs have been used in treating chronic hepatitis B and none have been proven to be effective, except interferon.

Treatment of HCV and HBV with interferon has limited success and has frequently been associated with adverse side effects such as fatigue, fever, chills, headache, myalgias, arthralgias, mild alopecia, psychiatric effects and associated disorders, autoimmune phenomena and associated disorders and thyroid dysfunction.

In all cases, a virus must enter a suitable host (plant, animal, bacterium), then transport successfully to a suitable host cell, enter the cell and undergo various cyclic changes mediated by the viral genome and viral proteins, enzymes and other viral biochemical's, some of which are synthesized within the virion particle and the majority of which are synthesized within the suitable host cell. Some plasmids and viroids exist which do not exhibit a virion, capsid or shell. However, most pathogenic virus' do exhibit some form of capsid, shell and overall capsid specificity. This invention can address inhibition during the formation stages for all of the aforementioned. Once successful in attaining the aforementioned milestones, a given unique virus present in a given unique host, has typically commenced passage from cell to cell, undergoing a filtering or screening effect. Clearly, all steps must occur, to then propagate progeny. It is the observation by researchers, of the presence of a lineage of progeny, which indicates a viral infection with undesirable side effect(s) which has been successful at pathogenicity. Further, it is observation of an accelerated deleterious effect, the morbidity and mortality imposed upon the cell, organ or host, which further indicates pathogenicity of a given virus. When the host exhibits signs that a pathogenic virus is at work, said virus can invariably be identified and acquired. Viral taxonomies exist, which provide a summation of desequencing efforts, cataloged logically, to demonstrate those naturally occurring pathogenic virus, sorted by their specific genomic peptide order, and organized also by region, typically enumerated by genes or gene regions. As such, there are two given prerequisites to help researchers further probe means to eliminate infection or to induce stable resistance to infection which are; the ability to recognize pathogenicity and tie it to a specific genomic sequence as found within a viral pathogen, and further; to demonstrate the range of specificity of each gene region, according to a viral taxonomy for said unique pathogenic sequence, as found in the genome of a virus causing deleterious, morbid and mortal effect upon a unique cell within a host and the unique host in question. Despite all of these known tools and processes available to those skilled in the art, a safe and universal solution to viral infection has remained illusive, and the full complement of drugs and biologicals proffered, cannot offer a safe and effective cure for these pathogenic virus and their deleterious effects, nor does the virus exhibit a stable form wherein these drugs and biologicals can trap the virus within a probable and reliable range of specificity, according to known and unknown taxonomy for said virus (good examples include HIV-1, or HCV/HEP-C).

In simple terms, the virus mutation capacity and recombination capacity is tested with various combinations of drugs and biologicals, and the virus will mutate or recombine (or both) to escape any and all attempts to fully eradicate the presence of same, from an infected host, or the infected host's cells. Quiescent forms of virus' are known to exist, which evade drugs, biologicals and combinations of both, to successfully escape therapeutic appliques and modalities. Drugs and biologicals are difficult to deliver to the internal portion of a cell, precisely where all virus' synthesize their progeny and thus replicate. As such, a limitation seems to be ever present, not only in terms of delivering a desirable therapeutic modality of drugs and biologicals to the internal portion of a cell, but also to render the drug and biological combinatorial effect, as an effect which completely eradicates the presence of the target pathogenic virus. One thing is certain. Viruses work, and they work well for their intended purpose.

Another observation is also certain. The sequence of a virus' genome, determines its range of function and potential for pathogenicity. There is a subtle, yet significant point to be made by accepting the fact that a given pathogenic virus, upon infection, is rapidly tailored through replication cycling, to attain perfection and achieve its intended purpose. A solution to a given virus for one seeking complete reversal of the pathogenesis, could logically take a similar form to the virus, with only subtle differences. The reasoning behind this premise is manifold. The most basic observation includes the acknowledgement that the virus is successful in completing many cycles of its replication, which closely parallels proliferation, deleterious effect upon living cells, organs and hosts.

### SUMMARY OF THE INVENTION

The preferred embodiment of the present invention is to properly acquire the starting sequence, properly analyze it, properly utilize predictive modeling for genome mutation and recombination potential, effectively isolate the promotors and terminators, fashion the genome in a replication incompetent format that will otherwise function in all areas such as infection, motility, folding, unfolding, post infection synthesis of viral biochemical constructs necessary to facilitate all of the aforementioned but not allow replication competence or full virion synthesis (mature, infectious virions), that is to create a theravirus, hereinafter referred to as “TheraVirus”. The basic approach of employing TheraVirus is a one-two concept where 1) competition is created with the target pathogenic virus with the object to break the replication cycle for same and reverse pathogenesis and improve markers, and 2) introduce any interfering construct which can directly effect other related viral genomes present in the same cell, at the same time and effectively render the whole cell incapable of producing virions. Should this be accomplished as described herein, using combinations of these techniques, these teachings will represent a revolution in virological technologies literally displacing biotechnology. The virus, that which causes all of the problems in hosts and host cells, also teaches us how to reverse its own pathogenesis. These simple observations strongly suggest what is believed to be the ultimate and most effective approach. These observations also lead to other logical conclusions. For example, constant TheraVirus viral loading and eventual reduction of the TheraVirus load occurs, where therapeutic benefits can be expected and a reduced target pathogenic viral load, in locked step with TheraVirus, when TheraVirus is optimally fashioned and applied. This means, TheraVirus could be proven effective for other forms of gene therapy while it provides relief and therapeutic benefit in an ever increasing and statistically significant populace. Cells cycle their internal chromosomal regions. It is believed this is an adaptation intended to clear viral nucleic acid sequences, plasmid or virion sequences or any other sequences which are not intended and are not human. As such, a constant load is the best form of gene therapy, if consistent performance comparable to the all inclusive function of natural chromosomes is the goal and objective. It is also believed that safety and performance are the factors that matter in fashioning human gene therapy that can be approved for common use and common good.

Clearly, there remains a need for an effective method of treating and prevention for viral infections. Additionally, the need exists in parallel, to perfect a gene therapy delivery tool, one that can be relied upon to remain stable and to deliver the genes needed by the recipient. If the cell indeed cycles out “vectors” and the like, so much the better. A patient can then upgrade delivery vehicles and rely upon the cell's cytoplasmic cycling (natural digestion) to clean up the mess, so to speak. Cell cycling has been observed in many organisms, including human cell studies. Cell cycling is observed during cellular replication, cellular repair, and cellular aging. Human chromosomes recode, according to aging. This is a spectacular issue because it reminds us, the use of gene therapy is not easily forecast as permanent and thus, experimentation with long, persistent loading of a suitable delivery vehicle would appear to be in order. There are logically two sources for the gene therapy; infecting or transfecting compositions. A whole virus, or a greatly reduced virus or plasmid (viriod). And these come in chimeric versions, trans-genetic variations and so the discussion can take on many variations. To load a cell with virions or plasmids, there is either a producer cell line deployed ex-vivo, or replication competence is used to provide the virions or plasmids in-vitro. (e.g. once placed in, infection is a cyclic and perpetuated event. In essence, this is self generated gene therapy.) The common denominator here is “perpetual loading”, which is an important feature of the TheraVirus teaching(s) of this invention. It must be remembered that the virus uses a strategy of constant loading and self perpetuation, to cause pathogenesis. This is a well known fact. TheraVirus seeks to mimic the effect, but not provide virions from within host cells, rather, from and external, controlled source.

### DETAILED DESCRIPTION OF THE INVENTION

The present invention borrows from nature, in that the composition is as closely modeled to the pathogenic source virus as possible. The composition is arguably not found in nature, simply because it includes known flaws which absolutely assure that the composition cannot replicate with an extremely high probability of maintaining replication incompetence, post insertion and for integrating viral genomes post integration, as well. Since natural pathogenic viral replication is the sole source for pathogenic virion presence within a host cell, organ or system (patient), the contemplated composition of this invention would not occur in nature beyond its one time mutated or recombined happenstance. It may be asked “Why model the virus from nature?” The answer is simple. The acquired sequence is known to work well, which means its capsid assembles properly, it hoists its genome into a matrix core, correctly, the virion matures properly indicating proper cleaving enzyme action and glycoprotein maturation. A myriad of intricate functions are in balance in a solid, reliable target pathogenic viral sequence. If an isolation of promotor and terminator is successful, and manipulation of same leaves the virion infectious, meeting all parameters of TheraVirus, but replication incompetent, one goal of the present invention is achieved. Thereafter, this sequence is used as a platform to deliver and activate specific genes, recoded to (for example) manufacture one, or perhaps a few faulty proteins. These proteins could reliably interfere with all known mutations of the protein producing gene in the pathogenic virus' taxonomy, hence, if the select protein were crucial in function, such as capsid shell synthesis, no capsid shells would form within an infected cell, if the faulty protein is expressed in great enough number and the rest of the “platform” remains reliable (reliably replication incompetent, reliably productive for only the intended byproducts).

The broad embodiment of this invention is to model a virus, nearly identical to its source pathogenic target. The composition must closely resemble the target or any variance the target is able to present. As such, the composition may be required in strains, just as the pathogenic varieties of retroviridae or viridae may occur in strains. The composition is considered for purposes of this invention, to be a mature, fully functional virion and the unique sequence of the delivered and integrated replication incompetent vDNA contained therein, or in the case of non-integrating RNA or DNA virus, a virion containing a replication incompetent genome. “Composition” is thus used interchangeably to mean either form (virion with genome or its genome, in its vDNA state or a genome in its RNA or DNA state [such as HEP-B associated cDNA]), and the context in which “Composition” is referenced in each sentence, will clarify to “one skilled in the art”, to which form we are referring.

The composition will provide for functional genomic regions which aid the proposed virions to follow the precise pathogenic pathway between cells and organs within a unique host. However, those genomic regions not needed for transport, infection and in the case of any subsequent transport, such as to the nucleus for purpose of integration of vDNA into a human chromosome, will specifically be rendered disabled. For most pathogenic viruses which include chromosomal integration (such as HIV-1 or HIV-2), it is not known if the vRNA, cDNA, vDNA or even a vPIC express regions, transcribe and translate proteins or enzymes prior to integration or prior to infection (successful fusion of the capsid to a host cell, delivering the viral genome as intended). It is believed that the capsid contains and carries enzymes, proteins and biochemicals synthesized from the prior host cell and the activities within a virion which further mature the virion, involve cleaving of longer proteins into differing glycoproteins, even after the virion has left the host cell. However, it is possible some viral genomic mediated change occurs in a virion, after it leaves its host cell.

Some viruses do not integrate their genome within a host cell's chromosome(s), but transport their genome to the nucleus within the nuclear barrier (membrane), and some viral genomes naturally stay outside of the nucleus electing to congregate vRNA at a ribosome, each according to their natural specificity. In any case, we are setting a standard of engineering and subsequent performance, to take (acquire) the pathogenic viral composition in question (each according to the TheraVirus teachings) and accurately predict its known pathway during transport within a virion (capsid), through successful and normal infection, carrying any needed viral mediated enzymes, viral mediated proteins and viral mediated biochemical's into the infection event, properly releasing the genome of the virus and its associated enzymes or other payload viral byproducts, to take the viral genome up to the point of replication competence and then, through specific and intended alteration of the genome's sequence (data), and in the case of the corresponding TheraVirus solution, leave the genome stranded as replication incompetent, no matter the extent or degree of mutation, or recombination opportunity. As such, a viral genome is delivered which merely (but effectively) “takes up space” and “exhibits the parallel and intended pathogenic cycling”, but falls short of replication. As discussed below, we anticipate each preferred embodiment to be a non-production coding for any and all genes which give rise to proteins, enzymes or other viral byproduct, used during the specific phase of pathogenic cycling, leading to virion production (no valid virion synthesis or no virion synthesis at all). For any virus, it is possible to delineate between expressions, chemical reactions and other biochemical activities which represent the infection phase, and delineate that phase indelibly, from the replication (virion synthesis) phase. Infection is an event which begins and ends for all viruses, virion by virion. So too, virion synthesis is an event which begins and ends, for all viruses, virion by virion. The invention thus focuses thus upon both the infection phase and the replication or “virion synthesis” phase, as the invention claims full infection competence and virion synthesis incompetence.

For all viral genomes, all promoters, terminators, start and stop codons (either by way of specific sequence alteration or upstream/downstream bp location relative to bp1 or combinations of the same) for genomic regions not involved in allowing the composition to successfully transport between cells, organs or hosts, and not involved in the process of infection through to successful chromosomal integration, will be candidates for alteration in a manner which is known to those skilled in the art (e.g. to halt expression, resequence or relocate the associated promoter). For pathogenic integrated vDNA constructs found in nature, the proper template alignment (3′ to 5′) and the reverse template orientation (5′ to 3′) must be taken into consideration when determining the genomic sequence for the composition and the contemplated changes, which subsequently disable targeted gene expression and transcription, or in the case of errant protein synthesis, start and stop codon rearrangement causing faulty viral protein synthesis or subsequent enzymatic cleavage of any synthesized viral protein, barring any incident of errant expression or transcription of the composition, or any recombination potential, leading to any composition driven attempt at virion synthesis. In addition, recoding of codons which directly relate to proteomic amino acid structures and sequences, will also provide for faulty protein folding, which in turn (in example) provide a prolific proteomic sub-unit which greatly impedes capsid formation if expressed, transcribed, translated and cleaved into a valid but faulty protein unit, thus folding to a state which, as it interacts with other viral proteomic subunits derived from other pathogenic viral genomes which are also present in a given unique cell, may exhibit hydrophobic bonding, electrostatic bonding or covalent bonding thus creating proteomic combinations which take up pathogenic viral proteomic subunits yielding complex proteomic bound units that cannot form a proper capsid, and instead yield a faulty capsid incapable of budding, or otherwise exiting the unique host cell. Contrary to the aforementioned preferred embodiment of “replication incompetence”, this specific embodiment argues for expression of perhaps one, or just a few viral genes. As such, and here again as “known to those skilled in the art”, we would require a promoter and terminator in proper upstream and downstream locale, to express only the intended gene region.

This is a secondary effect for the TheraVirus concept, which intends to leverage and exploit any success with the first composition. The ultimate target is the pathogenic version of the target virus. As such, no means exist to directly reach in and remove any and all natural pathogenic viral genomes from a unique host, or the byproducts of pathogenic viruses present in a unique host. Such potential is highly improbable. Instead, we seek to block and hinder the propagation of progeny of the target pathogenic virus through direct, safe and effective competition and at the same time, attempt to add to the interference factor, directly effecting those target pathogenic viral genomes synthesized subunits and other targeted viral pathogenic components, through limited production of our own controlled subunits and components which interact in such a manner as to bond with said pathogenic subunits or pathogenic components and halt their ability to continue to interact with other pathogenic subunits or pathogenic components in a manner which reduces their concentration. As such, the synthesis of pathogenic viral genomes, capsids, proteins, enzymes and the like, will thus be safely and reliably reduced.

The sequence for the composition will provide for as much identical molecular specificity as compared with the target pathogenic virus throughout its entirety, as is possible. But the elements defined must be resequenced in a manner that allows the composition to perform the tasks reliably, as aforementioned.

Once acquired, sequenced, packaged and tested, the composition is then produced homogeneously, within a suitable external producer cell line, as a valid, mature or rapidly and reliably maturing virion particle, which will mature and transfect as inserted into a suitable and unique host. Homogeneity between virion particles is important, as the specificity of the composition yields the desired effect and straying from the specificity of the composition, yields variable therapeutic effect to no therapeutic effect and at some point, loss of specificity during production could revert the composition back to a pathogenic form.

The compositions can also be used in conjunction with other treatments.

The composition is essentially, a valid and mature or maturing virion particle. The only difference between the composition and a pathogenic target virus, is the aforementioned alteration of promoter sequence or position, terminator sequence or position, start and stop codon placement, codon specific internal sequence manipulation, and codon by codon specific sequencing and order. Administration can vary and will range from direct injection, to possibly an inhaled composition or even a gel. Temperature, salinity, potassium level and other factors, such as presence of a water based storage means will all prove valuable to the successful storage and handling of the composition. The composition is HIV in essence (HIV represented here as but one preferred target pathogenic virus'), and so it is best stored in an environment that mimics human blood plasma, inclusive of a reasonable temperature range (eg human sanitized serum, temperatures maintained at 96° F. to 101° F.) Cryogenic freezing may be used to improve storage and “life expectancy” (shelf life, usually expressed as half-life)

The vDNA construct may be administered by any method known by one of ordinary skill, to be effective. For example, the composition of the present invention may be administered via intravenous injection or needle-less, noninvasive means.

This present invention relates to a therapeutic composition, in the form of a virus, fashioned directly from knowledge and acquisition of the genetic sequence of a given target pathogenic virus.

Disclosed herein, is a viral composition that inhibits the integration of targeted pathogenic vDNA subsequent to natural infection through systemic and systematic infection of cells within an animal. Although integration to plant chromosomes is not known, the same conditions exist in plants with respect to the presence of a natural genome and so, the same claim seems reasonable to uphold as a valid claim, e.g. the composition could inhibit the integration of targeted pathogenic viral genomes in plants, from an integrated chromosomal position. For purpose of illumination as a central theme and in one preferred embodiment, HIV-1, a recombinant Lentivirus of the Retroviridae class is discussed. Once “an individual skilled in the art” has reviewed this disclosure, it becomes clear how this teaching can apply universally, to all pathogenic viruses and not just those which integrate vDNA within human cell lines, as an integral phase of pathogenic viral replication. This teaching encompasses the formation of a composition which can cause the systematic, time based elimination of the pathogenic effect of a virus or phage within its preferred plant, animal and bacterium cell, or any virally infected host cell. This can be found in sources including but not limited to http://www.USPTO.gov, FDA, NIH, OBA, RAC, http://www.ASGT.org, http://www.retroconference.org, other publications of scientific abstracts and internet search engines such as http://www.AOL.com, http://www.altavista.com, and http://www.google.com, persistently reference the use of a virus (once attenuated), to act as a gene therapy vector or to induce immune system response. Never have been found reference to providing high titre, persistent loading thus creating a known, reliable infection rate in a unique host and the unique host's cells, to induce a therapeutic effect or to theorize a therapeutic effect. In essence, no one has yet proposed that a high titre’ of an attenuated virus or altered virus, can do more for a host cell, than take up resources and space. No one has proposed that slight modifications to a viral genome can yield predictable changes in said genome, rendering the genome replication incompetent for purpose of production of the replication incompetent format in question, subsequently applied to a host for purpose of safe and effective competition with a target pathogenic viral genome (virus), thus reducing a target pathogenic virus' virion production and potentially, eliminating the target virus virion production in situ.

The composition proposed is in fact, a working virus. The composition thus discloses close similarity to the embodiment of the target pathogenic virus, as represented by the composition's specific genome versus the pathogenic viruses genome, to which it is closely modeled against but distinctly and indelibly differentiated from the pathogenic genome by what may be as few as a 3-10 peptides difference, or perhaps 10-50 peptides difference, 50-150 peptides difference or may even presume the same overall molecular weight, carry the full complement of separable elements such as promoter, terminator, LTR, other genes but reorder said separable elements and then, introduce limited peptide manipulation. Wherein said target pathogenic virus and thus the starting template sequence, is representative of a long term surviving pathogenic viral product of its host (inclusive of a valid capsid thus forming a valid maturing or mature virion); Wherein consultation of a Viral Taxonomy to determine variations of a given sequence yields the range of variability of said sequence; Wherein said pathogenic virus is RNA or DNA based, Wherein said pathogenic virus is recombinant; Wherein said pathogenic virus is prone to mutation; Wherein the host exhibits the accepted markers for disease as caused directly or indirectly by the target pathogenic virus and hence, suffers deleterious effect from the target pathogenic virus' functional genomic consequence(s); Wherein said target pathogenic virus exhibits a given genetic sequence which can be acquired; Wherein said acquired sequence of interest, believed to be pathogenic and in fact being pathogenic exhibits Palindrome sequence(s), promoters, terminators, genes, introns, start codon sequences within expressed genes, and stop codon sequences within expressed genes; travels in the form of a virion, housed in its natural capsid, reliably transfects its targeted cells via receptors or coreceptors, forms cDNA and then vDNA, vPIC (Viral Preintegration Complex(s)) or RNA or DNA based genomes, and in the case of integrating genomes reliably integrates at a target palindrome site along a chromosome. Herein is disclosed a teaching which is derived from the target pathogenic viruses specific genome or the genomes derived from more than one lineage or strain of the target pathogenic virus, as primarily represented by their respective and highly specific genetic sequence data.

In one preferred embodiment, the composition will have to exist for each grouping of pathogenic target strain which exploits a unique palindrome target sequence or closely related palindrome target sequence and specific integration enzymes which code for the integration at these specific sites. In another preferred embodiment, RNA or DNA based genomes from non-integrating viruses will exhibit a similar engineering challenge requiring a different TheraVirus sequence for virions which contain a variable range genome, e.g. similar proteomic ladder proteins, or capsid shell proteins versus distinctly different proteins found in differing lineages within a unique host.

The viral composition in question, in one preferred embodiment of the invention, is a mature virion particle including capsid, inner membrane(s), nucleocapsid, viral enzymes, viral RNA, viral Proteins, viral Receptors and Co-Receptors, and is in all measurable ways identical to the matured virion form of the target pathogenic virus, but with minor alteration of specific portions of its internal genomic sequence. More specifically, for purposes of illustration, one preferred embodiment includes a completely normal, mature HIV-1 virion particle, containing all normal tangible and separable elements, such as proteins, enzymes and polypeptides including the characteristic twin RNA strands which would therein contain the alterations described herein, rendering the overall form of the virion a “composition”, as required for delivery of a therapeutic form of vDNA. However, upon successful infection, cDNA synthesis, vDNA synthesis, vPIC synthesis and movement to an integrated position at a targeted palindrome position within a chromosome, following the attraction of a given NLS (Nuclear Localization Signal); thereafter the “composition” is referenced as a successfully integrated vDNA composition, modeled according to teachings as herein provided.

Alteration of the start and stop codon sequences in genes which are normally expressed by the target pathogenic virus subsequent to successful, natural infection through natural pathogenesis, are proposed as preferred embodiments. Removal of the start and stop codon is obvious, to those skilled in the art, and is thus claimed as obvious, relative to these teachings. The same applies to codons used in synthesis of proteins and the reordering of these codons or even internal codon resequencing, to code for a different amino acid in the end product of translation. However, this invention intends to take into consideration the molecular weight and specificity of the pathogenic Viridae vDNA construct as found in the host, and maintain that specificity, preserving the fact that the starting sequence (template sequence for the pathogenic virus) vDNA in question is a long term survivor taken from a host suffering from unequivocal markers indicating viral initiated pathogenesis. The intent is to isolate the preferred starting pathogenic sequence, through acceptable scientific means known to those skilled in the art.

More specifically, through considerable sampling, isolation, desequencing and testing, to determine the highly successful and pathogenic strain in question and its best requisite sequence. By observation, that the target pathogenic virus in question is in fact, a closely paired and symbiotic virus as to the host (e.g., it has not yet killed the host in symbiosis with the condition that the host cannot clear the virus through any natural means, including the immune), the virus in question (sample) will have been “evolved” or “filtered”, as a function of time in the host, to work well for its intended purpose within the host.

For purpose of this invention, the intended purpose of a pathogenic viridae is to survive and to replicate as rapidly as possible. Admittedly, other purposes exist which include changing unique hosts effectively, to mutate frequently and to recombine. Said alteration (however fashioned), will cause the genomic regions in question to not express and thus not transcribe mRNA, subsequent to successful integration of the composition in its form of vDNA, for those regions which normally transcribe at this stage of a given pathogenic viridae integration. Said alteration taking into consideration proper alignment of the template strand of the vDNA upon successful Integration (3′ to 5′) and reverse Integration of the template strand of the vDNA (5′ to 3′) wherein the alteration of codon sequencia must hold true in any valid orientation and the alteration of promoter and terminator sequentia must equally hold true in any valid orientation. Said alteration taking into consideration the faulty nature of Reverse Transcriptase (viral enzymes) and thus expecting occasional mutation within the conversion from vRNA to cDNA and then to true vDNA, housed within a vPIC (Viral Pre-Integration Complex), the specific coding of the vRNA must alter more than one amino acid per intended change. An intended change for purpose of this invention, is defined as relocation of a codon, which is considered three amino changes for purpose of this invention, resequencing of a codon to no longer be interpretable as a start or stop codon, which requires more than one amino base (peptide) change per our definition (start and stop codons can read according to the first 2 of 3 peptides and so, careful consideration of this fact is noted), duplication of a codon or codons within a gene, relocation of any codon, resequencing of any codon, relocation of any promoter sequence, relocation of any terminator sequence, resequencing of any promoter sequence, resequencing of any terminator sequence, duplication of any promoter or terminator sequence, or recoding of any sequence or relocation of any sequence through change of more than one peptide in any given genomic region in question (or in any copy of a genome, such as the conversion of HIV vRNA to cDNA, or cDNA to vDNA, tracking to an integrated position within a human chromosome as but one requisite example) and taking into consideration proper insertion and reverse insertion (integration, alignment of the template strand) positions, which cause any sequence or relocation change to have two potential interpretations.

The viral composition, in its most preferred embodiment, is thus rendered or depicted as “replication incompetent”. Replication competence is not possible as a spontaneous event, because mutation or not, the mass majority of compositions reaching the integrated position within a valid chromosomal integration site (palindrome) will maintain a great percentage of the specificity intended throughout a statistically relevant number of concurrent infections, reverse transcriptions, vPIC formations, transportation and successful integration(s) performed by the homogeneous virions (compositions) and continue to hold true in a reverse integration, aligning the template strand in the 5′ to 3′ orientation. The same can be said for all viruses, as any virus has a genome and any viral genome can be altered thus. However, most do not integrate and so, the genome of a given pathogenic viruses still must follow its natural cycle, which can be an RNA or DNA based requisite construct which may or may not migrate to the nucleus of a host cell, according to its natural cycle and pathogenic activity.

It is clear, any natural pathogenic viral genome can be studied, altered and produced external to the preferred host cell, in a suitable producer cell line, and the homogeneity of the virions then produced, can be perfect or near perfect, one to the other. This is a technique known to those skilled in the art. As aforementioned, even though the technique is known, the claims for the use of the resulting virions have remained confined to development of live viral vaccines (immune system interacting) or vectors (gene therapy delivering). It should now be clear to those skilled in the art, that a given pathogenic viral genome can be altered to become replication incompetent, through study of the sequence data, acquisition of an understanding for key genes and their functions, key promoters and terminators and their proximity and location within the genome and the opportunities to selectively halt transcription of a given range of genes or genetic region(s), alteration of the sequence within those regions and selective resequencing or coding to render the composition “replication incompetent” according to the conventions of the target pathogenic virus. Furthermore, it is equally clear to those skilled in the art, that such a composition, once proven safe and effective, can be further enhanced through activation of but one or a few genomic regions or “genes”, through additional alteration of the successful genome, to yield a genome that meets the criteria for a TheraVirus of this invention.

Mutation is an issue which can be mitigated. In one preferred embodiment and as an example, too many start and stop codons will have been “disabled” in upstream or downstream direction and for any orientation (3′ to 5′ and 5′ to 3′ template strand integrated orientation [viral DNA plasmids are known to potentially integrate in both orientations and so, both orientations must be taken into consideration]) to allow any series of single point or multipoint mutations during reverse transcription and vDNA synthesis, to react with a latter (post integration) genomic stage of expression (transcription). Even if post integration genomic stages were mutated, and one or more did express (transcribe) post-integration, the intent is to provide in sum total (during the period of application), trillions of homogeneous virions (each containing the composition in question) on a continual basis, from external producer cell lines, into the unique host. The mass majority of these homogeneous and highly specific virions (e.g., 99.99999%) will maintain genomic specificity to the extent of non-expression of post integration genomic sequencia, even with mutation, to the extent that the composition will not enter into the target virus' replication cycle, or any hybrid form of viral replication cycle (chimeric or otherwise). The cell will then cycle out the composition as a function of time, and as a function of normal cellular replication (mitosis) and a continual loading (as aforementioned) of new, homogeneous compositions (virions) will assure the available sites for pathogenic viral integration within the subject cell (Palindromes), will again receive the replication incompetent vDNA composition with a very high probability, as directly relates to the loading scheme. The host cell which is infected is also believed capable of cycling out infected palindrome sites and “treated” palindrome sites. When new opportunities present to allow the composition's vPIC to compete with natural pathogenic virus' vPIC, the composition will win out through a higher overall loading scheme primarily, but equally important to consider are the issues of selecting a molecular specificity (overall) for the composition, which transfects well, is replication incompetent and yet, integrates well and is provided artificially through continual loading as sourced from a producer cell line with great homogenous specificity as to the absolute template sequence selected for the integrated form of the composition. Even if the natural cell cycles between cellular replication (periods of time absent cellular replication) and during cellular replication do not remove pathogenic vDNA, this approach still proves viable. Competition for integration will reduce pathogenic viral load and deleterious effect, as well as wasting effect(s). Energy and basic subunit concentration(s) will be enhanced and preserved because target pathogenic virion production must drop in the presence of a similar, competing composition formed as TheraVirus is formed. Lastly, pre-integrated pathogenic vDNA may well interchange with integration seeking plasmid rings formed by this composition, and to the extent molecular specificity, overall molecular weight or charge, or concentration of plasmids are concerned, we claim the ability to far out-compete natural target viral pathogenic plasmids seeking integration. Additionally, for those viridae which do not integrate vDNA, the same considerations are valid, for viral RNA based constructs seeking synthesis within the cytoplasmic realm and concentration at the appropriate organelles found within a cell (e.g. at a ribosome, or to interact with a limited amount of tRNA).

As to a living cell, such as a human cell, this prescribed viral composition is energy efficient and clearly limited in its commandeering of cellular energy. The viral composition will not produce mRNA post integration, for those genes selectively altered for non-transcription as described herein. Only through a remote probability of multi-point mutation, can a given gene again express, and the statistical opportunity for this event will be mitigated through changes in more than one amino acid within the sequence, including changes in all post integration expressed gene regions. In its most preferred embodiment, within start and stop codon sequence regions, at least (3) peptides will be substituted per gene (at least 2 per targeted codon and if a codon is a start or stop codon, the first 2 peptides as read in either orientation if the genome is a DNA genome). This greatly precludes mutation opportunity, per gene, to express mRNA within the targeted regions, post integration. This eliminates re-combinatorial concerns, as recombination is impossible without post integration expression and transcription. More importantly, expression of only one integrated viral gene within the composition in a given cell which is unintended, will not lead to virion production. This is known to “those skilled in the art”. Hence the odds of mutation within the conversion from mRNA to cDNA, prior to cDNA conversion to vDNA and then the migrational vPIC, mutation (such as that caused by reverse transcriptase) will occur approximately once in a base pair, every thousand base pairs. This guarantees a homogeneous lineage of composition as described herein, will survive all the necessary molecular steps to reach the integrated position within a chromosome of a living cell, and subsequently integrate (At a palindrome site, as intended). Integration of a replication competent mutant derived from the composition, would be probable and predictable, but will prove to be mathematically extreme in occurrence. It is roughly estimated one in 10,000,000 successful integration(s), or higher, for a replication competent mutant. Said composition, during any probable mutation, would still maintain natural specificity (could occur in nature). In any event, the cell cycle will clear all viral constructs integrated within the chromosomal material, and subsequent competition for integration will favor the described therapeutic composition. Should the cell cycle not clear these competing vDNAs, the same therapeutic effect is still a valid consideration and expectation. All of the aforementioned can be delivered in either integration orientation as viewed by measuring the orientation of the template strand of the composition, in its vDNA form. We simply need to examine, analyze and control the sequence in upstream and downstream orientations of the template strand, read from bp 1 through the last bp and then, taking the last bp as bp1 through to the beginning read in reverse but taken as a normal reading. By analyzing in both directions and making certain the sequence specificity codes as defined herein, replication incompetence and the other claimed features, remain intact for all orientations of the composition.

The favoring of the composition during integration is caused by the selected loading (selectively adjustable titre) and the overall specificity of the composition. The loading of the therapeutic virions must match the successful pathogenic virion plus a delimiter (increase over and above the titre of the target pathogenic virus) as determined by some experimentation, and contain only the minimum necessary change to specific genes, to assure overall near identical specificity. This is premised on the basis that the pathogenic viridae is carefully selected by the researcher for its successful replication competence and high rate of production as per its genome. The changes to this target viral sequence which results in the composition, will have to be selective to perform equally within proper integrated vDNA alignment and in its reverse integrated vDNA alignment (as per the template DNA strand, in a 3′ to 5′ orientation as proper and a 5′ to 3′ orientation taken to mean “as reversed”.) In this manner, viral protein production will be limited to pre-integration viral proteins attributable to this composition (which are derived primarily from the virion as it is naturally digested within the cytoplasmic realm, and few if any viral proteins are anticipated as “synthesized” for DNA genomes or reverse transcribing RNA genomes which form DNA until said genome reaches the point of post infection, entering into the cycle of virion synthesis and production). Post integration viral protein synthesis will be profoundly affected within the pre-infected host or within an uninfected host. The net sum total of cellular energy will thus be diverted back toward normal cellular function for the preinfected host cell, and thus the unique host, et al. Spare components, such as free amino acids or free lipids, will be conserved for other uses as the composition will not ravenously consume in great number, taking from the pool of these available subcomponents as the pathogenic vDNA's expression and transcription and translation would. Most importantly, at least two additional factors are anticipated. In an HIV+ patient with some thymic function and liver function still intact, maturation of immature immune system components will again appear normal subsequent to this form of therapy, and the overall immune system will be provided with an enormous opportunity of enhanced and extended time based co-existence with what was once a pathogenic viridae, to build cellular and humoral immune specificity and stable resistance from within a core of improving and restored immune system competence. Trec analysis and Immunoscope analysis of the resulting restored human immune system components (in humans, or even in a SCID-Hu Transgenic Mouse Model with human thymic and liver organ fragments and human immune system) are expected to return to normal or approach normal, quite rapidly in fact.

As used in low, medium or high titre loads (doses), this composition will impart the entitled effect upon the targeted cell which is prone to infection and pathogenesis as caused by the target pathogenic virus. Loading schemes will vary depending upon circumstance and some experimentation will be required, however, using those tools known to researchers “skilled in the art”, the composition in question can be loaded dynamically, and titred according to the titre of the target pathogenic virus, where the host is pre-infected with said target virus. For purpose of this acquisition of the titre of the pathogenic virus and the composition, probing techniques known to “those skilled in the art” will be applied to plasma, blood plasma, mucus, cytoplasm, nucleoplasm and even individual chromosomes. To determine valid titre for molecular immunization in those not pre-infected with the target pathogenic virus, a reasonable threshold similar to the target pathogenic virus will prove to be effective. Higher titres are believed utterly safe and so, the loading for molecular immunization can vary, according to the wishes of the protocol in question. There will however, be a certain minimum threshold titre which must be maintained, in order to impart effective, long term molecular immunity. This may also vary from protocol to protocol. In all cases, probing techniques known to those skilled in the art, can detect the successful placement of these compositions within the target cells, as described within “In Situ Hybridization and Probing Techniques”, according to the current state of the art. As such, the protocol threshold can be achieved for each and every pathogenic virus, each and every patient and thus add an additional dimension of safety and effectiveness for all patients, or potential “at risk” hosts to enjoy. The researcher can start with a loading scheme of double the titre of the pathogenic viridae, measure PCR, RTPCR, use in situ hybridization techniques or even direct fluoroscopically marked and electron microscope filmed titre acquisition for the pathogenic viridae and the composition in question, to determine the necessary loading scheme over the entire intended observation period. As aforementioned, we can look inside the fluids, cells, nuclei and chromosome, to prove our data and the comparison of pathogenic viral loads to the composition's load, as a function of change in either over time. This can implicate daily testing as aforementioned, weekly testing and perhaps monthly testing, to determine the daily dose of the composition. The composition will definitely be required daily, and possibly twice per day, to maintain the targeted and selected titre for the composition.

The composition is compatible with all other known therapeutic modalities. The composition causes no undesirable side effect in the target host cell.

For a host that can recover from secondary and tertiary effects of all prior exposure to the pathogenic virus in question, with the entitled effect administered to the cells in question, the morbidity, mortality and wasting of the cell, organ and host is reversed and eradicated.

It should be abundantly clear and apparent to those “skilled in the art” that the teachings herein described, represent a method which can be deployed against any pathogenic virus. In broad embodiment of the invention, it is proposed to mimic a pathogenic strain, with a nearly identical strain, but increase the titre of the competing strain while making certain the competing strain does not synthesize any unnecessary proteins or other genetic, genomic or proteomic constructs (such as mRNA), in order to maintain immunosilence and not to provoke a myriad of other pathogenic consequences as normally posed by the targeted pathogenic viridae. As used herein, the term immunosilence refers to the prevention of an intefering immune response or a deleterious immune response. The competing strain is the composition. Its titre is artificially maintained as “high”, relative to the target pathogenic virus. Its specificity is homogeneous (each virion is identical to the next and to the intended composition model, to a very high degree, inclusive of its functional sequence). The composition's presence, interaction with the host and the composition's viral functions which we allow to remain as “naturally expressed”, compete with the target pathogenic virus. As this applies to HIV-1, a retroviridae of the Lentiviral class, a recombinant virus, the competition sought is for integration within human chromosomes, within CD4+ cells and other human cells, identified by type and subtype. The final effect is to greatly reduce natural pathogenic HIV's integration probability. The composition, as properly administered and thus creating the intended titre, systematically reduces target pathogenic viral protein synthesis, recombination opportunity, mutation in terms of “measurable effect” probability and opportunity, greatly conserves cellular energy and thus breaks the replication cycle for the targeted natural wild-type pathogenic HIV strains.

It is known that virions can be loaded into a system, such as a human system, in any concentration and that the immunologic and pathogenic outcome is based upon either a) protein product specificity of the virion, or b) any proteins or byproducts produced thereafter, as the virion transforms through its natural pathogenesis and c) Statistically relevant observation from prior patients, with respect to the specific strain of the virus in question. The embodiment of the present invention is a virion, which is created within a suitable producer cell line and thus, includes a total viral genome encased within a valid, active and mature or maturing virion, which can be loaded at almost any level, into a target host cell, organ or system (animal, plant or bacterium) and not cause a deleterious effect, while at the same time, allowing for certain controlled activities to take place, through the regions of expression (promoted and terminated regions) and through codon manipulation, e.g. start codon or elimination of same, stop codon or elimination of same or multiple duplications of any such element.

A virion which does not follow through with high production of post transformation constructions, as it moves from virion to RNA or DNA activities, is far less likely to promote immunological or pathogenic activity in a host, or more specifically, to a host cell in the case of immunologic activity and within a host cell, in the case of pathogenic activity. Virion replication is a highly complex and highly evolved and symbiotic event, particularly in mammalian cells and more specifically, human cells. Because the process is so finely tuned to human cell metabolism(s) and human “system” metabolism(s), the desired effect of therapeutic function is easily created in a number of ways using the means of the present invention. In brevity, we believe a replication incompetent virion which closely matches a successful pathogenic virion, is generally going to prove to be immunosilent, if the means of removing replication competence reside within quashing, suppressing and down regulating everything “genetic and genomic” within the virion in question, in order to continue to maintain all normal function of the virion and its genomic content only up to a very specific point in its cycle, while maintaining overall molecular specificity for each separable component of the pathogenic virion. At some point in the linear and logical cycle of natural pathogenic virion to viral RNA or viral DNA synthesis, for any virus, there is a stopping point which can be determined by specific sequence manipulation. This lineage of virions has a very high probability of successfully competing with the target pathogenic virion, either through integration as herein described, or through competition at the sites of synthesis, such as at the ribosome. Interference can include blocking in the real sense, or production of faulty proteins which cannot travel far, do not leave the cell, are not toxic and are recycled by the cell's natural enzymes intended to cycle cytoplasmic contents and promote efficiency.

HIV, HEP-B and HEP-C are characterized and noted by researchers as having deleterious effect which is associated with persistent presence and high viremia states within the target host and within target host cells. Which is to say, high titres for pathogenic viral byproduct within the blood plasma or sample cells taken from the host. However, virion replication leads to biological byproduct in measurable places. Blood plasma or cellular contents are good places to take the measurements and draw correct conclusions as to the presence or absence of a given virus and its current load. There are many “probing based” measurement techniques including PCR and RTPCR (Polymerase Chain Reaction), or “in situ hybridization” techniques, as well as blotting, molecular weight separating effects and the like. Hence, any safe and non-deleterious means to eliminate virion production from within a living cell without introducing any deleterious effect to the cell and prove it through these measurements (or other reliable viral load and activity measurement techniques), is a therapeutic modality.

All known antiviral therapy theories (which follow accepted FDA positive indicia for antiviral therapeutic markers) focus on reductions in viral load as a therapeutic marker, without variance or question. As this invention uniquely proposes, virion replication can be eliminated through competition. This can be induced safely, through introduction of a competitive virion lineage. For safety and to follow a logical course, it is believed a replication incompetent format is preferred. It is possible to create a virus that replicates slowly, blocks target pathogenic virus' and remains stable. However, this is undesirable because there are billions of processes which could alter the genome of the “therapeutic virus” in question. Cells cycle their chromosomal expressed region contents. This slows the progression. This is one reason why HIV cannot kill in days, weeks or months. The immune system is not able to control HIV-1, generally. 98% of HIV-1 infected individuals will inevitably succumb to AIDs. This is disputed in various papers, so lets accept 75% for purpose of discussing this invention. The statistically significant evidence is still overwhelmingly convincing that the human body fights a long, hard fight against HIV, and in 20 years of research, it does not appear that the immune system is truly the source of the resistance. Instead is appear to be more a function of the cycling of the regions where HIV can source and commence virion production. It is believed the organelle structure, such as the microtubule, and the successful navigation of that organelle, gaining access to the nucleus by a “large molecule complex” also represents a very important barrier which mitigates the pace at which HIV+ becomes AIDs. These compositions are vDNA inserted into human chromosomes, which are first mitigated by probability and statistics. This is the first weakness of the virus that the current invention intends to exploit. The fact that HIV does not reach the chromosome but one in twenty attempts. If the therapeutic virion (composition) can be loaded, to a very high titre, this must result in a higher probability of “integration”, provided my virion is integration competent.

Integration for HIV is a multi-part symbiotic event. Viral Integrase, catalysts and helper molecules abound in the process. Palindromes are involved. The sites for integration are thus limited to a palindrome which interacts with viral or human restriction and integration enzymes and their catalysts or other helper molecules; and thus integration for a pathogenic strain of virus is thus limited to those sites which are addressable as aforementioned. Integration is not spontaneous or accidental. Integration is also rare as heavy molecules do not reach the nucleus of a mammalian cell with frequency, particularly in human cells. HIV has adapted to form, transform and transform again, to travel from individual human host to human host, cell to cell and even within the cell. Subsequent to successful integration, comes the expression and successful transcription of mRNA, its successful “trimming” and export from the nucleus, to the golgi apparatus, massing ultimately at a virion budding site. A bazaar of gene swapping takes place (recombination) post integration, along with a fairly frequent and reasonably predictable mutation probability during reverse transcription, pre-integration.

In another embodiment of the invention, is a method wherein all HIV-1 associated codons, promotors and terminators not needed for virion maturation, transport, fusion, digestion, release of viral enzymes and release of dual RNA strands, allow reverse transcriptase to function, allow reverse transcription, synthesize cDNA, synthesize vDNA, synthesize the vPIC and transport the vPIC to the nucleus of the preferred target cell(s). Provided is an HIV virion that is completely functional as the pathogenic version and it is desired only to alter specific genetic sequences found as subsequently expressed and thus translated within the integrated vDNA composition. TheraVirus, in a preferred embodiment keyed to pathogenic versions of HIV-1, thus takes the form of an HIV virion particle with specific changes in the integrated vDNA. HIV virion, which is viable, which goes through its complete cycle to the point of integrating a vDNA composition. It is the composition that matters, once integrated. It is the fact that the virion is homogeneously manufactured externally and loaded persistently. It is the fact that the vDNA delivered, can only integrate based upon a sequence of highly dependent events which include response to NLS (Nuclear localization signals), proper function of viral integrase, presence of and proper function of numerous catalysts, proper exploitation of human and viral palindromes, creation of a plasmid ring, controlled lysing of the ring and the human chromosome at the palindrome and ultimately, a clean and reliable integration.

vDNA integration is likely to be a mutually exclusive site by site event. vDNA likely integrates once per palindrome. Even though two identical palindromes are created by the one integration, viral Integrase looks upstream and downstream from the palindrome. Once vDNA is integrated, the two palindromes created have different upstream or downstream amino acid sequences containing the palindrome. Thereafter, HIV vDNA does not sequentially integrate or chain in a linear fashion. For example, vDNA in 2, 3, 4 or many fold linear and sequential alignments multiplying in linear fashion at the palindromes the first vDNA integration creates for example, bp 1 through 9899 and again 1 through 9899 and so on. It is expected that vDNA inserts of this composition, will not permit integration of an adjacent upstream or a downstream pathogenic vDNA integration (exploiting the palindrome at each end of the successfully inserted composition). If this is incorrect, then the TheraVirus composition will do the same thing (linear chaining). And so, the conceptual competition with natural pathogenic vDNA(s) will take place and the TheraVirus concept will prove to be therapeutic. If pathogenic HIV integration probability is one in twenty, this method changes it to one in 100 or even better, favoring less pathogenic vDNA integration and thus less pathogenic viral genomic function, overall. Pathogenic HIV must proliferate, or the immune system can respond effectively and the immune system thus contains the potential to reverse pathogenesis. The overall probabilities and statistics issues sway heavily in favor of TheraVirus. TheraVirus can thus hedge out pathogenic HIV because HIV is so very dependent on occasional breakout.

The inserted vDNA is now in question. HIV virions function as known to those skilled in the art. The inserted vDNA can contain man made sequences, a fact also known to those skilled in the art. However, the prior art does not disclose the idea of competition and persistent loading to change the probabilities and statistics associated with pathogenic viral vDNA integration potential and subsequent pathogenic effect as caused by that potential. For that matter, the opposite thinking holds true. The prior art does not disclose the idea of interruption of the probabilities and statistics associated with pathogenic viral vDNA integration potential and subsequent pathogenic effect as caused by that potential, can break the replication cycle for the targeted pathogenic virus and thus reverse pathogenesis. In its most preferred embodiment, this implies a total cure can be achieved. By turning off expression of all genes involved after Integration of the composition in question as defined herein, it is possible to show that the method of the invention is predictable and safe.

First, the changes to the vDNA as dictated by changes in the RNA brought in successfully by the TheraVirus virion would include isolation and disabling of the targeted promotor regions. Since chromosomes are read in only one direction, there is a terminology used of downstream and upstream. However, vDNA can be inserted in its correct orientation or in a reverse orientation as a natural and expected potential. As such, all discussion of upstream and downstream directions must imply the direction from 3′ to 5′ or 5′ to 3′ for the template strand. To bar the expression of any post integration gene region within the composition, it would be necessary to remove promoters. More importantly, it is necessary to add terminators. Removing a promoter is easily done, however it is also necessary to maintain overall molecular specificity. Thus, substitution of a terminator into a promoter region, may well serve the intended purpose. Total molecular specificity is in question, as is proper operation of the composition subsequent to each proposed change. Thus it is clear, some experimentation is needed, although the outcomes are easily predicted. For example, in removing a promoter by substituting a terminator into the promoter position, the gene targeted for expression by the now removed promoter, will not express and thus will not transcribe any mRNA. By placing a reliable terminator just prior to the gene, in the former promoter position, a dual effect may be achieved. Promotion will not occur for that gene from the prior promoter (now removed) and any errant promotion may also terminate just prior to the gene in question, guarding against any errant or stray promotion caused by a source considered to be outside of the gene region in question. In a preferred embodiment included is a terminator sequence prior to every gene expression region which is used to synthesize virions, post integration. Therefore, the RNA delivered by the TheraVirus virion reverse transcribes to form cDNA, vDNA, vPIC, a plasmid ring and inserts properly and reliably at a palindrome position within a targeted chromosome, as attracted by a NLS.

Each gene contains codons. Start and stop codons do not define the beginning and end of a gene. They define areas for protein cleaving, much further along in the mRNA to ribosome and tRNA interactions. The idea here is to disable proteins through skillful manipulation of codons. In this manner, if TheraVirus vDNA was promoted and one or more genes transcribe mRNA, the mRNA will contain faulty but safe, start and stop codon sequences. The anticipation is for no such coding at all. The reality is that human or other unrelated viral processes could potentially provide an integration and promotion of the composition's genes at a future date. Thus the logic behind codon manipulation becomes clear to those skilled in the art. Even if all the genes in the composition were expressed and transcribed, the subsequent proteins produced would retain maximum length and specificity and would not cleave reliably, thus virion production would not occur for the pathogenic virus from which the composition was taken. Nor would these products predictably or reliably align with an unrelated virus, which arrives prior to or subsequent to the introduction of this composition for therapeutic effect.

The overall specificity of the TheraVirus virion, its contents and ultimately, its RNA, must function as a pathogenic HIV virion, with respect to fusion, reverse transcription, vPIC formation, mobility and integration. Thereafter, the more immunosilent the composition can be, the less we transcribe, and if we transcribe, the less compatible the proteins are with pathogenic viral synthesis or any other unrelated viral synthesis, the better off this overall approach will be as to providing a safe and reliable therapeutic effect.

The host is used as a genetic filter by a pathogenic virus. Virions that are appearing and proliferating, are demonstrating through their absolute molecular and genetic specificity, that they are compatible with the host in question. The host is alive at that point and the immune system is not perfectly effective, but there is a balance and thus, a symbiosis. So the virus and the host are compatible and the virus is proliferating, which means the virus is effectively leveraging the host's cells, organelles within those cells and the natural genomic function(s) of those cells, demonstrating said symbiosis and a dynamic advantage as to virus versus host. As such, there has to be a teaching in the specific sequence of the successful and prolific pathogenic virion's genome. Accessing a viral taxonomy can assist in demonstrating the target sequence§ range, and where the viral sequence in question resides within a taxonomic analysis. However, the sum total of the message and teaching here is “This is a compatible genome, which proliferates well in this human”. Since solid scientific research has reliably identified functional genetic structures like promotor regions, terminator regions, genes, codons and the like, this invention applies a logical set of teachings to:

a. Devise a virion (composition) that will mimic the pathogenic targeted virion and leverage the same elements, which are many, and are very well tuned to the support of the pathogenic version of the virion in question;

b. Turn off all “virion synthesis elements” within the composition but leave all others present or alternatively, selectively turn on virion synthesis elements, but fashion them as faulty, relative to the extent of interfering with other competing and expressing viral genomes present within the host cell;

c. Not have undue toxic or immunologic effects;

d. Traverse the regions of bloodstream, lymphatic, various cell types, various receptors and so forth, with identical reliability as to the pathogenic form of the targeted pathogenic viral strain;

e. Create a replication incompetent format while maintaining all other elements and maintain awareness that vDNA can and will integrate in a normal and in a reversed orientation relative to the target chromosome. It is also possible viral DNA could form an unexpected integration which is not defined by merely stating the normal or reversed orientation, rather, loops, hairpins and the like. No matter what the orientation, the polypeptides will be read in one direction or in another and all interpretive expression or transcription and any errant translation, will follow the guidelines set forth herein; and

f. Rely on all the aforementioned to then load the host with a greater load of these virions as opposed to the real time titre for the targeted pathogenic virus in question;

Natural Integration of vDNA is not quite difficult to plan or execute. Even for the naturally occurring pathogenic versions of the virus in question (HIV), its a rare event. But once accomplished, billions of virions can be produced from an integrated position. This holds true because the progeny of the cell are producers of HIV, either through inheritance of vDNA within a chromosome, or the “jump” of virions which occurs as the cell membrane pinches off and intakes a considerable amount of external plasm, along with the virions in question. Even the stray vPICs in the mother cell, can be predictably expected to migrate to the daughter cell as the daughter cell pinches off from the mother, during a successful cellular replication cycle. If HIV, as but one example, is drastically reduced in its virion production, simply because a competitor arrives which greatly changes the odds for integration, then the competitor (TheraVirus) will have proven to be therapeutic. TheraVirus will be predictably therapeutic because the competitor is muted or down regulated, in its use of cellular energy and cellular components like amino acids, by way of the competitors limited use of protein production, mRNA production and at the same time, expression of the human chromosome upstream and downstream from the insertion, should remain normal. The present invention envisions taking the virus from the host. Whole chromosomes are paramount to have functional at all times. If the virus in question had been suppressing the chromosome of its host, this could be detected prior to utilizing the sample virion through means known to those skilled in the art, and if this activity was present in a considerable number of cells within the host, it can be asserted that the cells would be suffering more obvious deleterious effect, as well as the host.

It is the genetic and proteomic filtering of the host, which produced the virion which this invention utilizes, i.e., dissects to acquire a definitive sequence, match it to a database (as a purely logical routine), and then inflect the changes to the vDNA which are defined herein. Thus, the net product is known to be compatible and borrows from the patient's genetic and immunologic tolerance and tailoring of the pathogenic virus in question.

Tests upon cells are anticipated, checking outside and inside of the cells, nuclei and even chromosome(s) for viral load data and non-transcription, as predicted within this provisional application. This testing can proceed along the accepted guidelines of the scientific method and for that matter, in accordance with all published guidelines for this type of work, as published by recognized authorities. We will test cells, SCID Hu mice which can host a human immune system, complete with human organ fragments, chimpanzees will be added (simian/primate) and interestingly, we can select animals from other failed experimentation and see if a therapeutic effect can be prompted. Throughout this process we will fine tune a human protocol and consider human trials if and when the data is statistically relevant and indicative of a risk reward benefit equation.

Drugs and biologicals normally require a very long period of research and testing to lead to bona-fide discovery of an efficacious modality. Viruses tend to mutate or recombine beyond any and all applicability of drug or biological combination therapies and most particularly, HIV. The goal of the present invention is to change this lineage of restriction and limitation by way of the TheraVirus concept discussed above.

What is claimed is:

1. A method for constructing a virus that is a competitive inhibitor of HIV viral pathogenesis for the treatment of HIV comprising, obtaining a replication incompetent HIV-derived virus, wherein the replication incompetent virus replicates in an external producer cell line to form a virus particle comprising the replication incompetent viral genome and proteins required for infection and integration of the replication incompetent virus into a host cell genome, wherein the replication incompetent virus is obtained by the steps of:
(a) mutating an HIV-derived virus such that at least 3 amino acid residues per gene are mutated;
(b) determining that the virus of step (a) infects the host cell;
(c) determining that the virus of step (a) integrates into the host cell genome;
(d) determining that the virus of step (a) does not replicate in the host cell.

2. The method of claim 1, wherein the virus is mutated in step (a) by the insertion of terminator sequences into promoter regions.

3. The method of claim 1, wherein the virus is mutated in step (a) by the deletion of promoter sequences.

4. The method of claim 1, wherein the virus is mutated in step (a) by point mutations.

5. The method of claim 1, wherein the virus is mutated in step (a) by mutating start and/or stop codons.

6. The method of claim 1, wherein the virus is mutated in step (a) by mutating at least 2 nucleic acid residues in a codon.
